(19) United States
(12) Patent Application Publication
(10) Pub. No.: US 2016/0037811 A1
(43) Pub. Date: Feb. 11, 2016

(54) FRUIT AND VEGETABLE PRODUCT FOR CURING ORAL ULCER, AND PREPARATION METHOD THEREFOR

(71) Applicant: GUANGZHOU BOXABIOTECH CO., LTD., Guangdong (CN)

(72) Inventors: Tiancun Xiao, Guangzhou (CN); Renju Chen, Guangzhou (CN); Li Li, Guangzhou (CN); Pengfei Zhang, Guangzhou (CN)

(21) Appl. No.: 14/778,964

(22) PCT Filed: Sep. 13, 2013
(86) PCT No.: PCT/CN2013/083471

§ 371 Date: Sep. 21, 2015

(30) Foreign Application Priority Data
Apr. 17, 2013 (CN) 201310135167.4

(51) Int. Cl. A23L 1/212; A23L 2/02; A61K 36/73; A61K 36/31; A61K 36/03; A61K 36/21; A61K 36/81; A61K 36/42; A61K 36/18; A61K 35/747; A61K 36/88; A61K 36/8905; A61K 36/23; A61K 35/748; A23L 1/30

(52) U.S. Cl. CPC A23L 1/2121; A23L 1/3014; A23L 2/02; A23L 1/30; A61K 36/73; A61K 36/31; A61K 36/03; A61K 36/21; A61K 36/81; A61K 36/42; A61K 36/18; A61K 35/747; A61K 36/88; A61K 36/8905; A61K 36/23; A61K 35/748; A23V 2002/00; A23Y 2220/15; A23Y 2220/67

(57) ABSTRACT

A fruit and vegetable product for curing oral ulcer, and a preparation method thereof. The fruit and vegetable product is made by mixing the following crude materials of different parts by weight: vegetables and/or fruits of 30-100 parts, food additives of 0-15 parts, probiotics powder of 0-5 parts, and recipients of 0-40 parts, the vegetables and fruits being cool and cold, cool and mild, and cold and mild; and the vegetables or fruits being cool, cold or mild in the theory of traditional Chinese medicine.

### TECHNICAL FIELD

The present application pertains to the field of food technology, especially fruit-vegetable based food for treating oral ulcer and method for making the same.

### BACKGROUND

Oral ulcer is a superficial ulcer that occurs in the oral mucosa. According to the theory of traditional Chinese medicines, oral ulcer is caused by internal heat rising in the liver and stomach channels, as well as those caused by hypoimmunity or deficiency of trace elements, are mainly treated by diverting the excessive internal heat. Nowadays, drugs commonly used for treating oral ulcer include watermelon cream and Kangfuxin solution. Watermelon cream mainly helps in heat-relieving and fire-purging. Kangfuxin solution applied in local spraying treatment, which is significantly higher than watermelon cream treatment. The mechanism of Kangfuxin solution is to facilitate growth of pelleting tissue, enhance anti-inflammation, eliminate inflammatory edema, improve body immunity, and the like.

However, the exact cause of oral ulcer is not yet completely clear. It is widely believed in the field that it is the result of multiple factors combined, with a complicated mechanism that has not yet reached a final conclusion.

Therefore, a therapeutic formulation with significant efficacy by combining the strengths and advantages of Chinese medicines and modern nutrients for treating oral ulcer is urgently needed.

### SUMMARY OF THE INVENTION

Accordingly, one aspect of the present disclosure provides fruit-vegetable based functional foods for treating oral ulcer which present multiple advantages in treating oral ulcer and with significant overall efficacy.

In an embodiment of the aforementioned aspect, the following technical solution is provided:

Fruit-vegetable based food for treating oral ulcer, consisting of:

| | |
|---|---|
| vegetable and/or fruit | 30~100 parts by weight; |
| food additive | 0~15 parts by weight; |
| probiotics | 0~5 parts by weight; |
| excipient | 0~40 parts by weight; |

wherein the vegetable and/or fruit are vegetable and/or fruit considered in theories of Chinese medicines as of cool nature and cold nature, cool nature and mild nature, cool nature and cold nature and mild nature, cool nature, or cold nature.

The term “vegetable” refers to plants and fungi, except for grains, that can be cooked into food. There are various methods for treating oral ulcer with complicated mechanisms, which, to summarize, treat oral ulcer mainly by reducing continuous ulceration of the ulcer surface and facilitating forming of new tissues. Therefore, the present application treats oral ulcer by sterilizing and clearing extra-heat from the ulcer surface as well as supplementing active substances that facilitate growth of new tissues. According to theories of traditional Chinese medicines, vegetables and fruits of cool nature and cold nature have the effects of clearing fire-heat from body. Vegetables and/or fruits contain antioxidative nutrients (e.g., vitamin E) that may prevent peroxidatic reaction of unsaturated fatty acids, stabilize cytomembrane. Other nutrients contained in vegetables and/or fruits, along with probiotic additives, may promote body immunity, energy metabolism, cell viability, and body resistance, as well as improve local circulation, so as to enhance healing of the ulcers. In addition, vegetables and/or fruits of mild nature may be added to vegetables and/or fruits of cool nature and cold nature to achieve a balance and better therapeutic effects, in accordance to the integrative conditioning theories of traditional Chinese medicines. Types and additive amounts of various ingredients are designed by taking into account the nutrient contents in the raw materials in computational simulation. Particularly, vegetables and fruits containing various nutrients that fulfill the daily needs of all populations are chosen and formulated in appropriate proportions according to Chinese Dietary Guidelines: Recommendations for Trace Element Intake.

In some embodiments, the fruit-vegetable food aforementioned is comprised of the following ingredients by weight:

| | |
|---|---|
| vegetable and/or fruit | 30~70 parts; |
| food additive | 2~5 parts; |
| probiotics | 2~5 parts; |
| excipient | 30~40 parts. |

In some embodiments, the vegetables and/or fruits are comprised of the following ingredients by weight:

| | |
|---|---|
| powder of vegetable and/or fruit | 30~95 parts; |
| extract of vegetable and/or fruit | 0~5 parts. |

The extract of vegetables and/or fruits above is formulated according to target components and concentrated from vegetable and fruit materials. Specifically, appropriate extraction methods are employed according to the different nature of various target components, e.g., solvent method with selected solvents according to the polarity of target components. Microwave extraction, ultrasonic extraction, steam distillation, sublimation method, squeezing method, supercritical extraction, or any other modern techniques may be used in extraction.

In some embodiments, the vegetable and/or fruit of cool nature or cold nature includes at least one selected from the group consisted of: pear, watermelon, banana, grapefruit, melon, mulberry, strawberry, kiwi, water chestnuts, asparagus, radish, rape, spinach, red beet, eggplant, tomato, cherry tomato, spinach, rape, water spinach, bitter gourd, cucumber, celery, mung bean, lotus leaf, kelp, laver, wakame, volvox, and spirulina; the vegetable and/or fruit of mild nature includes at least one selected from the group consisted of: apple, cabbage, parsley, and Chinese cabbage. Vegetables and fruits of cold or cool nature have outstanding healing effects against oral ulcer caused by excess fire, accumulation of damp heat in intestines and stomach, etc. In addition, vegetables and fruits contains abundant vitamins and minerals, especially vitamin B, which may have good auxiliary heating effects for oral ulcer caused by deficiency in nutrients. Such vegetables and fruits also include abundant polyphenols that help recovery cytomembrane, activate immune cells, promote comprehensive immunity, and improve defense against diseases. Addition of mild vegetables and fruits further allows a more balanced and enhanced nutrient spectrum for the vegetable-fruit food.

In some embodiments, the vegetables are vegetables with greater than or equal to 0.1% glucosinolates in their dry weight. Such vegetables mainly include cabbage, carrot, Chinese cabbage, white radish, and other crucifers. Glucosinolate is a sulfur-bearing secondary metabolite that widely exists in rape, cabbage, mustard, radish and other crucifers.

Thioglycoside is a stable compound, but it may degrade into multiple degradation products if catalyzed by bacteria enzymes in the gastrointestinal tract. Glucosinolate and its degradation products have active biochemical activities that protect immune cells in intestines and have antioxidative and anticancer effects. Therefore, when vegetables rich in glucosinolate are added, the vegetable-fruit food is endowed with functions of regulating digestive system. Modern medical theories believe that regulating gastrointestinal conditions may promote growth of intestinal villus, increase the villus length of small intestine, ileum, and duodenal, and increase mucosa thickness of the small intestine. As a result, growth of intestine bacteria is facilitated, which improves the intestinal environment. Intestinal tract movement is also enhanced, which increases absorption of nutrients by the intestines. A protection layer is also formed as a result on the stomach wall that prevents gastric acid from stimulating and eroding the stomach wall and thus prevents gastrointestinal inflammation. Recovery of gastric mucosa is also promoted. The vegetable-fruit food contains rich dietary fiber, which in turn may promote intestinal movement and growth of bacteria enzymes, increase absorption of nutrients, and help removing stool and toxins. Dietary fiber also facilitates removal of harmful microbe in intestines and growth of intestinal microbial communities, conditioning the intestinal environments. Furthermore, the vegetable-fruit food is usually rich in anti-ulcer vitamins K1, U, and B as well as glutamic acid and arginine. Vitamins K1, U, and B may protect and recover gastric mucosa, maintain the activity of gastric cells, reduce chances of gastric ulcers, and prevent alcoholism. Glutamic acid and arginine may promote growth of small intestine villus. Therefore, the vegetable-fruit food described herein may effectively improve conditions of low immunity and stomach fire caused by deficiencies in trace elements, dietary fiber, specific vitamins, and other nutrients. In addition to oral ulcer, it also has good effects in regulating oral diseases such as ozostomia and other diseases such as chronic gastritis, stomach ache, gastrectasis, gingivitis, gum bleeding, constipation, and the like.

In some embodiments, the probiotics include at least one selected from the group consisted of: Bifidobacterium adolescentis, Lactobacillus Bifidobacterium, Bifidobacterium bifidum, Lactobacillus crispatus, Lactobacillus acidophilus, Lactobacillus casei, Bifidobacterium longum, Bifidobacterium breve, Lactobacillus acidophilus, Lactobacillus bulgaricus, Lactobacillus fermenti, Lactobacillus gasseri, Lactobacillus helveticus, Lactobacillus johnsonii, Lactobacillus plantarum, Lactobacillus reuteri, Lactobacillus rhamnosus, Lactobacillus salivarius, and Streptococcus thermophilus.

In some embodiments, the food additive includes at least one selected from the group consisted of: flavoring agent, essence, and thickener; the excipient includes at least one selected from the group consisted of: filler, lubricant, and adhesive. The flavoring agent includes sweetening agent and acidulant. Various natural essences may be added according to needs (e.g., attract children), which may add different tastes to the vegetable-fruit food, such as mint, fruit, vanilla, or milk flavor. Lubricants and adhesives may be added according to conventional formulation requirements.

In some embodiments, the flavoring agent includes at least one selected from the group consisted of: citric acid, vitamin C, tartaric acid, white sugar, stevioside, xylitol, and aspartame; the essence includes at least one selected from the group consisted of: menthol-like flavor, strawberry flavor, banana flavor, pineapple flavor, melon flavor, and vanilla flavor; the thickener is carboxmethyl cellulose; the filler includes at least one selected from the group consisted of: maltodextrin, microcrystalline cellulose, lactose, tapioca starch, and corn starch; the lubricant includes at least one selected from the group consisted of magnesium stearate and talcum powder; the adhesive is syrup.

A method for preparing the fruit-vegetable food for treating oral ulcer as described herein is provided, comprising: selecting the above-mentioned vegetable and/or fruit; adding food additive, excipient, and probiotic powder; and mixing evenly to obtain the fruit-vegetable food. Preferentially, the vegetable and/or fruit are/is processed into powder.

The preparation method described herein combines processing methods of raw materials from traditional Chinese medicines and modern techniques, such as spray drying, ultrafine grinding, vacuum freeze dehydration, and the like. The unwanted enzymes against nutrients in the raw materials are thus inactivated, their influences on weaker people thus reduced. Meanwhile, it improves the nutrient release rate and efficiency, which in turn enhances the target efficacy. The methods of the present application employ advanced processes of dehydration, extraction, and concentration, such as supercritical carbon dioxide extraction and spray dry techniques, to further extract effective components, reduce moisture content, and inactivate enzymes. As a result, the expiration date of the materials is prolonged and the target nutrients more concentrated. Furthermore, the vegetable and fruit powders are subject to ultra fining which yield powders of 100 mesh or smaller, which allows the effective components to be even easier to release and get in touch with the digestive system.

In some embodiments, the method further comprises a step of preparing the fruit-vegetable food into forms of tablets, electuary, solid drink or vegetable paper, which allows convenient and immediate use.

The present application has the following advantageous effects compared to prior art:

The vegetable-fruit food for treating oral ulcer provided herein integrated various strengths in traditional Chinese medicines, with ingredients beneficial for human health that may clear fire-heat as well as facilitate ulcer healing, treating oral ulcer in a comprehensive method. In experiments of treating oral ulcer, the cure rates of the products provided herein were as high as 96%, with a significant overall healing effect.

Furthermore, when vegetables rich in glucosinolates are added to the vegetable-fruit food as described herein, they may provide a synergistic effect along with other nutrients and regulate the digestive system. In experiments of treating chronic gastritis, the overall effective rates were as high as 100%, higher than 76% of the control group.

The vegetable-fruit food provided herein contains no pharmaceutical components, chemical preservatives, or any other unwanted additives. It is completed made from daily vegetables and fruits, as well as natural food additives, without any transgenic crops. Therefore, unlike drugs, there is no consumption limit or any toxic effect for human body for the vegetable-fruit food provided herein, which thus makes a suitable long-term dietary therapy for patients suffering from uneasiness in digestive system and oral cavity. In addition, it may also act as a nutritious supplementary for residents in undeveloped areas lacking vegetables and fruits, infants, elder people, and people with busy work and irregular or unbalanced diets.

A method for preparing the vegetable-fruit food for treating oral ulcers as above is also provided herein, which combines processing methods of raw materials from traditional Chinese medicines and modern techniques, such as ultrafine grinding which improves absorption efficiency and nutrient composition, which further improve the efficacy.

### DETAILED DESCRIPTION OF PREFERRED EMBODIMENTS

Detailed examples are described below for purposes of illustration, without limiting the scope of the invention in any aspect.

### EXAMPLE 1

Tablets of fruit-vegetable foods for treating oral ulcer are prepared in this example, comprising the following ingredients by weight:

| vegetable and | apple powder | | 30 | parts |
|---|---|---|---|---|
| fruit powder: | cabbage powder | | 30 | parts |
| | kelp powder | | 10 | parts |
| | spinach powder | | 10 | parts |
| vegetable extract: | cabbage extract | | 1 | part |
| food additives: | essence: | strawberry essence | 0.2 | part |
| | | menthol | 0.06 | part |
| | flavoring agent: | stevioside | 0.09 | part |
| excipient: | lubricant: | talcum powder | 1 | part |
| | | magnesium stearate | 0.05 | part |

| | | | |
|---|---|---|---|
| filler: | lactose | 4 | parts |
| | maltodextrin | 10 | parts |
| | tapoica starch | 3.6 | parts |
| adhesive: | 60% sucrose solution | 20 | parts. |

The method for preparing the fruit-vegetable food above comprises:

1. Selecting vegetables and fruits of good quality and considered as of cool nature, cold nature, or mild nature in traditional Chinese medicines, such as apple, cabbage, kelp, and spanich;

1.1 Grading

The edible part of the vegetables and fruits to be processed are graded and undersized, damaged, or rotten individuals are discarded;

1.2 Cleaning

Dusts, dry leaves, litter and the like are washed off the vegetables and fruits to be processed with running water;

1.3 Sterilizing

The cleaned vegetables and fruits are sterilized and the enzymes are inactivated with hydrogen peroxide, chlorine, ultraviolet rays, infrared rays, gamma rays, and the like.

2. Extract the effective constituents of the vegetables and fruits

2.1 Drying and Cutting

The sterilized vegetables and fruits may be dried in the oven under 100° C. or less, by natural sun light, or in the shades. Drying under lower temperature allows better protection of the nutrients in the vegetables and fruits. The vegetables and fruits are dried until its moisture contents is lower than 10% and then cut into pellets no more than 0.5 cm in diameter.

2.2 Secondary Drying

The dried pellets are dried for 48 hours in the oven under 100° C. until the water content is between 3%-5%, which are then sealed in packing bags complying with food standard and stored under room temperature.

2.3 Pulverizing

The dried leaves or fruit pellets are ground to fine power using superfine grinding technology with a 100-mesh pulverizer. The feeding speed and rates are controlled to prevent jamming.

2.4 Preparing Cabbage Extract

Cabbages are extracted with conventional solvent extraction method (e.g., see Tu et al. 2007. The Extraction Conditions of Glucosinolates from Leaf Mustard Journal of food science and biotechnology 26: 9-12). Cabbages are immerged in ethanol and heated in order to extract. The solvent is then recovered in order to obtain cabbage extracts.

3. An appropriate amount is obtained from each kind of dried powers of vegetables and fruits as well as other ingredients, in accordance with the recipe above, by weighing using an electronic scale.

4. Mixing

All the weighed ingredients (except for adhesive and lubricants) are combined and mixed evenly.

5. Sterilizing

The mixture from step 4 is sterilized with gamma ray so as to ensure that the bacterial content meets the GMP process criterion. 6. Preparing Adhesive (Syrup)

An appropriate amount of purified water is weighed in accordance to its proportion in the recipe, heated to about 75° C., and stirred until a corresponding amount of white sugar is dissolved therein.

7. Wet Pelleting

Operators, wearing gloves made of clean materials that comply with standards for direct food contact, slowly add the adhesive syrup to the well-mixed semi-finished product from step 5 while stirring. The pelleting criterion requires the product to be aggregatable by holding yet scatterable by touching.

8. Screening

Screening the pellets from step 7 with sieves of 20 mesh and 70 mesh, respectively. Pellets that may pass 20 mesh but not 80 mesh are selected to meet the manufacturing requirement of tablets.

9. Pellet Drying

The semi-finished product from step 8 is dried in the oven under 55° C. for 3 hours, until the water content is less than 5%.

10. Adding Lubricant

Talcum powder and magnesium stearate of appropriate proportions are added to the mixture and mixed evenly.

11. Tabletting

The tablet press is set with proper pressure and filling rate, cleaned, and activated to make tablets.

12. Testing

The manufactured tablets are checked for their surface smoothness, rigidity, brittleness, homogeneity and integrity of its appearance, and metal contents. Unqualified tablets are removed.

### EXAMPLE 2

A solid drink of fruit-vegetable food for treating oral ulcer is provided herein, comprising the following ingredients by weight:

| | | | |
|---|---|---|---|
| vegetable and fruit | cherry tomato powder | 10 | parts |
| powder: | water melon powder | 10 | parts |
| | pear powder | 20 | parts |
| | kiwi powder | 20 | parts |
| | bitter melon powder | 30 | parts |
| probiotics: | Lactobacillus bulgaricus | 5 | parts |
| food additives: | essence: menthol | 0.05 | part |
| | flavoring agent: stevioside | 0.05 | part |
| excipient: | adhesive: 60% sucrose solution | 25 | parts. |

The method for preparing the fruit-vegetable food comprises:

1. Material Selection

Selecting vegetables and fruits of good quality and considered as of cool nature or cold nature in traditional Chinese medicines, such as cherry tomato, water melon, kiwi, pear, and bitter melon.

1.1 Screening

The edible part of the vegetables and fruits to be processed are graded, undersized, damaged, or rotten individuals are discarded;

1.2 Cleaning and Cutting

Dusts, dry leaves, litter and the like are washed off the vegetables and fruits to be processed with running water. Inedible parts are removed, and the edible parts are cut to pieces within 1 cm in diameter.

2. Processing into Powder

2.1 Pulping/Juicing

The raw materials are pulped or juiced depending on the material characteristics. Materials subject to pulping include cherry tomato, kiwi, and bitter melon. Materials subject to juicing include water melon and pear.

2.2 Filtering

The pulped or juiced materials are filtered to remove large-grained suspended solids.

2.3 Concentration

The vegetable-fruit pulp or juice is concentrated by heating and stirring which vaporize excessive moisture.

2.4 Adding Supplementary Materials

Supplementary materials such as maltodextrin in appropriate proportions are added to the vegetable-fruit pulp or juice and stirred until uniform to increase the solid content and facilitate drying.

2.5 Spray Drying/Vacuum Freeze Drying

The fluid-like material is spray dried or vacuum freeze dried, sterilized, and processed into powders, with moisture content controlled under 5%.

2.6 Packing

The vegetable-fruit powders are collected and packed according to the specification with packing materials of food grade.

2.7 Testing

The packages are checked for leakage, damage, and presence of metal pieces more than 0.5 mm in diameter.

2.8 Storage of the semi-finished products of vegetable-fruit powders

The powder-like semi-finished products are stored under 15° C. or less, in shaded, dry, and dark conditions.

3. Material Weighing

An appropriate amount is obtained of each kind of materials in accordance with recipe above by weighing.

3. Mixing

All the weighed ingredients are combined and mixed evenly.

4. Pelleting

Adhesives are added and pellets are manufactured by automatic pelleting production process using a high-speed pelletizer.

5. Drying

The materials are cold dried under 60° C. or less until its water content is less than 5%.

6. Sterilizing

The mixed materials are sterilized using γ ray at 3kGy for about 2 hours to kill bacteria and viruses.

7. Packing

The pellets are packed with packing bottles of food grade in sizes of 50 g per bag and 10 bags per pack.

8. Code Spraying

The product package are sprayed on their surface with codes indicating date, manufacturing lot number, and the like, using a code machine.

9. Product Storage

The products are stored under a temperature of 15° C. or less and humidity of 50% or less, and in dry, shaded, vented, and dark conditions.

### EXAMPLE 3

A liquid drink of fruit-vegetable foods for treating oral ulcer is provided herein, comprising the following ingredients by weight:

| | | | |
|---|---|---|---|
| vegetable and | red beet | 25 | parts |
| fruit powder: | tomato | 10 | parts |
| | strawberry | 18 | parts |
| | melon | 10 | parts |
| | cucumber | 15 | parts |
| | radish | 10.5 | parts |
| food additives: | thickener: CMC (carboxymethyl cellulose) | 0.5 | part |
| | sweetening agent: white sugar | 10 | parts |
| | acidulant: citric acid | 1 | part |

The method for preparing the fruit-vegetable food comprises:

1. Material Selection

Selecting vegetables and fruits of good quality and considered as of cool nature or cold nature in traditional Chinese medicines, such as red beet, tomato, strawberry, melon, cucumber, and radish.

1.1 Grading

The edible part of the vegetables and fruits to be processed are graded, undersized, damaged, or rotten individuals are discarded;

1.2 Cleaning and Cutting

Dusts, dry leaves, litter and the like are washed off the vegetables and fruits to be processed with running water. Inedible parts are removed, and the edible parts are cut to pieces within 2 cm in diameter.

1.3 Precooking

The cut-up vegetable-fruit pieces are precooked under 60° C. for 10 minutes.

2. Processing into Vegetable-Fruit Juice

2.1 Juicing

Vegetables and fruits with low pectin contents, such as red beet, melon, cucumber, and radish are juiced using continuous juicing method; while vegetables and fruits with high pectin contents, such as tomato and strawberry, are juiced with a beater with 0.7 mm sieve pore size. The juice is centrifugally filtered using canequim and diluted with water at 1:1 ratio by stirring for 10 minutes. The materials are juiced again and the secondary juice is mixed with the previous juice product to improve juice yield.

2.2 Flash Sterilizing

The vegetable-fruit juice from the steps above is rapidly heated to 100° C. and rapidly cooled after 1 minute.

2.3 Spray Drying/Vacuum Freeze Drying

The sterilized fluid-like material is spray dried or vacuum freeze dried, sterilized, and processed into powders, with moisture content controlled under 5%.

2.4 Liquid Production

An appropriate amount of the vegetable-fruit powder is weighed according to the recipe proportions and dissolved in water at a ratio of 1:5. Supplementary materials such as sweetening agent, acidulant, and thickener are added to the vegetable-fruit juice to improve the product flavor and homogeneity.

2.5 Hot Canning

The vegetable-fruit juice is heated under 80° C. for 30 minutes for heat sterilization, immediately sealed in cans in sizes of 350 ml per can, and quickly cooled down.

2.6 Testing

The packages are checked for tightness and content clarity.

### EXAMPLE 4

A powder of fruit-vegetable foods for treating oral ulcer is provided herein, comprising the following ingredients by weight:

| vegetable and fruit powder: | banana powder | | 6 | parts |
|---|---|---|---|---|
| | water chestnut powder | | 6 | parts |
| | apparatus powder | | 6 | parts |
| | celery powder | | 6 | parts |
| | spirulina powder | | 6 | parts |
| probiotics: | Lactobacillus plantarum | | 5 | parts |
| food additive: | flavoring agent: | citric acid | 1 | part |
| | | stevioside | 0.05 | part |
| excipient: | filler: | maltodextrin | 14.95 | parts. |

The method for preparing the fruit-vegetable food comprises:

1. Material Selection

Selecting vegetables and fruits of good quality and considered as of cool nature or cold nature in traditional Chinese medicines, such as banana, water chestnut, apparatus, celery, and spirulina.

1.1 Grading

The edible part of the vegetables and fruits to be processed are graded, undersized, damaged, or rotten individuals are discarded;

1.2 Cleaning and Cutting

Dusts, dry leaves, litter and the like are washed off the vegetables and fruits to be processed with running water. Inedible parts are removed, and the edible parts are cut to pieces within lcm in diameter.

2. Processing into Powders

2.1 Pulping/Juicing

The raw materials are pulped or juiced depending on the material characteristics. Materials subject to pulping include banana, apparatus, celery, and spirulina. Materials subject to juicing include water chestnut.

2.2 Filtering

The pulped or juiced materials are filtered to remove large-grained suspended solids.

2.3 Concentration

The vegetable-fruit pulp or juice is concentrated by heating and stirring that vaporize excessive moisture.

2.4 Adding Supplementary Materials

Supplementary materials such as maltodextrin in appropriate proportions are added to the vegetable-fruit pulp or juice and stirred uniform to increase the solid content and facilitate drying.

2.5 Spray Drying/Vacuum Freeze Drying

The fluid-like material is spray dried or vacuum freeze dried, sterilized, and processed into powders, with moisture content controlled under 5%.

2.6 Packing

The vegetable-fruit powders are collected and packed with packing materials of food grade in size of 5 g/pack.

2.7 Testing

The packages are checked for leakage, damage, and content uniformity.

### EXAMPLE 5

### Comparative Experiment of Effectiveness in Treating Oral Ulcer

### I. Materials and Methods

1. General Information

250 patients suffering from recurrent oral ulcer, including 150 males and 100 females of 30-60 years old with an average age of 42, were selected. Selection criterion: Selected patients suffer from recurrent oral ulcer with a seizure frequency of at least once a month, appearing in single or multiple locations in circular or oval shape with mild congestion in the surrounding tissues. The ulcer surface is yellow or white in color, accompanied with burning pain. The recurrence is frequent and at various time. Various other infectious stomatitis and Behcet's disease are excluded. Oral ulcer may be graded according to WHO grades: grade I, painful white or red spotty ulcers; grade II, white or red spotty ulcers, with solid food acceptable; grade III, ulcers, with only liquid food acceptable; and grade IV, bleeding, with no food form acceptable. Accordingly, the patents include: grade I: 75 cases; grade II: 125 cases; and grade III: 50 cases. The 250 patients were divided into treatment and control groups, with no statistically significant differences in their gender, age, primary disease, conditions, oral ulcer grades, and numbers of focus, and therefore comparable.

2. Experimental Methods

Patients in the control group took three Sanlian tablets (Beijing Sanlian Pharmaceuticals Co., Ltd, Health permit: (Beijing Drug) Health and Food Certificate (2006) No. 110000-JS0003) three times a day. After 8 courses of treatments, each consisted of 6 days, the patents were followed up for half a year. Patients in the treatment group 1 took three tablets made as in Example 1 three times a day. After 8 courses of treatments, each consisted of 6 days, the patents were followed up for half a year. Patients in the treatment group 2 took a pack of vegetable-fruit solid drink made as in Example 2 three times a day. After 8 courses of treatments, the patents were followed up for half a year. Patients in the treatment group 3 took 100 ml vegetable-fruit drink made as in Example 3 three times a day. After 8 courses of treatments, the patents were followed up for half a year. Patients in the treatment group 4 took a pack of vegetable-fruit powders made as in Example 4 three times a day. After 8 courses of treatments, the patents were followed up for half a year.

3. Criterion of Efficacy

Cured:

Short term: The ulcer surface disappears completely and mucosa recovers to normal after one course of treatment.

Long term: Less than one seizure in the half-year follow-up period.

Significantly Effective:

Short term: The ulcer surface reduced significantly, but a small area of ulcer surface is still present.

Long term: 2-4 seizures in the half-year follow-up period.

Ineffective:

Short term: The ulcer surface did not change.

Long term: More than 5 seizures in the half-year follow-up period.

II. Results

The experimental results are listed in Table 1 below. Compared to the control group, the treatment groups had significantly higher efficacy in their effect in treating oral ulcer:

TABLE 1
Results for oral ulcer treatment

| | | Number | | Significantly | | Overall | Cure |
|---|---|---|---|---|---|---|---|
| Group | | of Cases | Cured | Effective | Ineffective | Efficiency | Rate |
| Control | Short | 50 | 36 | 12 | 2 | 96% | 72% |
| Group | Term | | | | | | |
| | Long | 50 | 6 | 12 | 32 | 36% | 12% |
| | Term | | | | | | |
| Treatment | Short | 50 | 48 | 2 | 0 | 100% | 96% |
| Group 1 | Term | | | | | | |
| | Long | 50 | 42 | 8 | 0 | 100% | 84% |
| | Term | | | | | | |
| Treatment | Short | 50 | 47 | 3 | 0 | 100% | 94% |
| Group 2 | Term | | | | | | |
| | Long | 50 | 42 | 8 | 0 | 100% | 84% |
| | Term | | | | | | |
| Treatment | Short | 50 | 46 | 4 | 0 | 100% | 92% |
| Group 3 | Term | | | | | | |
| | Long | 50 | 44 | 6 | 0 | 100% | 88% |
| | Term | | | | | | |
| Treatment | Short | 50 | 46 | 4 | 0 | 100% | 92% |
| Group 4 | Term | | | | | | |
| | Long | 50 | 45 | 5 | 0 | 100% | 90% |
| | Term | | | | | | |

Table 1 shows that, the short-term overall efficiencies of the control group and treatment groups all exceeded 96%, with the treatment groups having obviously better curing effects. However, the long-term overall efficiency and cure rate of the control group were merely 36% and 12%, respectively, while those of the treatment groups were 100% and greater than 84%. The manufactured products taken by the treatment groups were able to facilitate rapid healing of the ulcer surface, especially refractory, recurrent oral ulcer, and showed better effect and advantages in postponing the seizures and complete healing. This is because the products described herein comprises not only the same heat-clearing, antibiotic components as the other products for treating oral ulcer, but also rich minerals, diet fiber, vitamins A and B, and the like, which condition the sickness from inside the body and increase body immunity. In addition, the tablets produced as in Example 1 contain rich vitamins U and K that may heal ulcers and stop bleeding. The various formulations prepared as described herein all had outstanding therapeutic effects in treating recurrent oral ulcer without significant different among them.

### EXAMPLE 6

### Comparative Experiment of Effect in Treating Chronic Gastritis

I. Materials and Methods

1. General Information

Patients suffering from chronic gastritis were divided into five groups as follows: control group of 50 patients, 35 males and 15 females with an average age of 46.8 (24˜60) years old and average course of disease of 8.9 (1˜23) years, including 25 superficial gastritis, 18 atrophic gastritis, 5 erosive gastritis, and 2 bile reflux gastritis; treatment group 1 of 50 patients, 33 males and 17 females with an average age of 45.3 (25˜60) years old and average course of disease of 9.7 (2˜22) years, including 26 superficial gastritis, 16 atrophic gastritis, 5 erosive gastritis, and 3 bile reflux gastritis; treatment group 2 of 50 patients, 30 males and 20 females with an average age of 45.1 (25˜60) years old and average course of disease of 9.6 (2˜22) years, including 28 superficial gastritis, 15 atrophic gastritis, 5 erosive gastritis, and 2 bile reflux gastritis; treatment group 3 of 50 patients, 32 males and 18 females with an average age of 45.2 (25˜60) years old and average course of disease of 9.2 (2˜22) years, including 26 superficial gastritis, 16 atrophic gastritis, 6 erosive gastritis, and 2 bile reflux gastritis; treatment group 4 of 50 patients, 31 males and 19 females with an average age of 45.4 (25˜60) years old and average course of disease of 9.7 (2˜22) years, including 25 superficial gastritis, 18 atrophic gastritis, 5 erosive gastritis, and 2 bile reflux gastritis. There were no statistically significant differences (P>0.05) among these groups in terms of gender, age, course of disease, and types, and thus comparable. Selection criterion: \textcircled{1} irregular stomach pain and fullness, epigastric fullness or uneasiness, especially after eating, usually accompanied by nausea, vomiting, flatulence, acid reflux, appetite decrease, and yellowish fur; \textcircled{2} Examination: mild epigastric tenderness; \textcircled{3} endoscopic examination showed various degrees of congestion spots, edema or erosion in gastric mucosa, red and white in color or color changes in the gastric mucosa, thinner and smooth plica, and exposure of veins.

2. Treating Methods

Patients in the control group took a pack of Weikangling granules three times a day for 3 courses of treatments, each consisted of 7 days. Patients in the treatment group 1 took three tablets made as in Example 1 three times a day for 3 courses of treatments, each consisted of 7 days. Patients in the treatment group 2 took a pack of solid drink made as in Example 2 three times a day for 3 courses of treatments, each consisted of 7 days. Patients in the treatment group 3 took 100 ml of vegetable-fruit liquid drink made as in Example 3 three times a day for 3 courses of treatments, each consisted of 7 days. Patients in the treatment group 4 took a pack of vegetable-fruit powders made as in Example 4 three times a day for 3 courses of treatments, each consisted of 7 days.

3. Criterion of Efficacy

Cured: Cardinal symptoms disappear, active inflammation in mucosa disappear as determined by gastroscope, chronic inflammation recovers to mild degree, gland atrophy is shown by gastroscope and confirmed by biopsy pathology, gastric acid substantially recovers to normal, and bile reflux disappears.

Significantly Effective: Cardinal symptoms substantially disappear, active inflammation in mucosa substantially disappear as determined by gastroscope, chronic inflammation improves, gland atrophy is shown by gastroscope and confirmed by biopsy pathology, and significant improvement in gastric acid functions and bile reflux.

Effective: Cardinal symptoms significantly alleviate, extent of disease in mucosa reduces no less than ½ as determined by gastroscope, inflammation alleviates to some degree, alleviation of chronic and acute inflammation (at least one grade) is shown by gastroscope and confirmed by biopsy pathology, and improvement in gastric acid functions and bile reflux.

II. Results

The experimental results are listed in Table 2 below. Compared to the control group, the treatment groups had significantly higher efficacy in their effect in treating chronic gastritis:

TABLE 2
Results for chronic gastritis treatment

| | Number | | Significantly | | | Overall | Cure | % Significantly |
|---|---|---|---|---|---|---|---|---|
| Group | of Cases | Cured | Effective | Effective | Ineffective | Efficiency | Rate | Effective |
| Control | 50 | 24 | 12 | 11 | 3 | 94% | 48% | 24% |
| Group | | | | | | | | |
| Treatment | 50 | 28 | 20 | 2 | 0 | 100% | 56% | 40% |
| Group 1 | | | | | | | | |
| Treatment | 50 | 28 | 21 | 1 | 0 | 100% | 56% | 42% |
| Group 2 | | | | | | | | |
| Treatment | 50 | 26 | 22 | 2 | 0 | 100% | 52% | 44% |
| Group 3 | | | | | | | | |
| Treatment | 50 | 27 | 21 | 1 | 0 | 100% | 54% | 42% |
| Group 4 | | | | | | | | |

Table 2 showed that the overall efficiencies of the control group and treatment groups all exceeded 90%, with the treatment groups having obviously better curing effects. Particularly, the cure rate and percentage of significantly effective were significantly higher in the treatment groups than the control group. The various formulations prepared as described herein all had outstanding therapeutic effects in treating chronic gastritis without significant different among them.

### EXAMPLE 7

### Comparative Experiment of Effect in Treating Ozostomia

I. Materials and Methods

1. General Information

Patients of ozostomia were divided into five groups as follows: control group of 50 patients, 36 males and 14 females; treatment group 1 of 50 patients, 35 males and 15 females; treatment group 2 of 50 patients, 33 males and 17 females; treatment group 3 of 50 patients, 36 males and 14 females; and treatment group 4 of 50 patients, 35 males and 15 females. The average age is 24˜48 years old. Selection criterion: Halimeter value greater than 50 ug/L as determined by a portable digital breath analyzer; exclusion of oral malodor caused by uremia, diabetes, and hepatopathy, other cardia-cerebrovascular disease, and pregnancy. There were no significant differences among these groups in terms of gender, age, and course of disease.

2. Treating Methods

Patients in the control group used 20 ml Sanjin Watermelon cream mouthwash for 30-second mouthwash to wash all corners of the oral cavity once in the morning and once in the evening, without eating or rinsing for 30 minutes afterwards, successively for 7 days. Patients in the treatment group 1 took three tablets made as in Example 1 three times a day successively for 7 days. Patients in the treatment group 2 took a pack of solid drink made as in Example 2 three times a day successively for 7 days. Patients in the treatment group 3 took 100 ml of vegetable-fruit liquid drinks made as in Example 3 three times a day successively for 7 days. Patients in the treatment group 4 took a pack of vegetable-fruit powders made as in Example 4 three times a day successively for 7 days.

3. Criterion of Efficacy

Significantly Effective: Ozostomia symptoms disappear.

Effective: Ozostomia symptoms alleviates to some degree.

Ineffective: Ozostomia symptoms do not change.

II. Results

The experimental results are listed in Table 3 below. Compared to the control group, the treatment groups had significantly higher efficacy in their effect in treating ozostomia:

TABLE 3
Results for ozostomia treatment

| | Number | Significantly | | | Overall | % Significantly |
|---|---|---|---|---|---|---|
| Group | of Cases | Effective | Effective | Ineffective | Efficiency | Effective |
| Control | 50 | 6 | 32 | 12 | 76% | 12% |
| Group | | | | | | |
| Treatment | 50 | 28 | 22 | 0 | 100% | 56% |
| Group 1 | | | | | | |
| Treatment | 50 | 30 | 20 | 0 | 100% | 60% |
| Group 2 | | | | | | |
| Treatment | 50 | 27 | 23 | 0 | 100% | 54% |
| Group 3 | | | | | | |
| Treatment | 50 | 29 | 21 | 0 | 100% | 58% |
| Group 4 | | | | | | |

Table 3 showed that the control group and treatment groups all had therapeutic effects to some degree. The overall efficiency and percentage of significant effective cases were 100% and more than 54%, respectively, for the treatment groups, while the percentage of Significant effective was only 12% and the Ineffective was 24% for the control group. The various formulations prepared as described herein all had outstanding therapeutic effects in treating ozostomia without significant different among them.

The detailed embodiments described herein are only for the purpose of illustrating the present invention, and are not intended to limit the scope of the present invention in any way. It would be understand by a person skilled in the art that various changes and modifications can be made to the embodiments described herein without departing from the scope and spirit of the present invention. Such changes and modifications are contemplated by the present invention, the scope of which should only be defined by the following claims.

1. Fruit-vegetable food for treating oral ulcer, comprising of:

| | | |
|---|---|---|
| vegetable and/or fruit | 30~100 | parts by weight; |
| food additive | 0~15 | parts by weight; |
| probiotics | 0~5 | parts by weight; |
| excipient | 0~40 | parts by weight; |

wherein the vegetable and/or fruit are vegetable and/or fruit considered in theories of Chinese medicines as of cool nature and cold nature, cool nature and mild nature, cool nature and cold nature and mild nature, cool nature, or cold nature.

2. The fruit-vegetable food of claim 1, comprising of:

| | |
|---|---|
| vegetable and/or fruit | 30~70 parts by weight; |
| food additive | 2~5 parts by weight; |
| probiotics | 2~5 parts by weight; |
| excipient | 30~40 parts by weight. |

3. The fruit-vegetable food of claim 1, wherein the vegetable and/or fruit are comprised of:

| | |
|---|---|
| powder of vegetable and/or fruit | 30~95 parts by weight; |
| extract of vegetable and/or fruit | 0~5 parts by weight. |

4. The fruit-vegetable food of claim 1, wherein the vegetable and/or fruit of cool nature or cold nature comprises at least one selected from the group consisted of: pear, watermelon, banana, grapefruit, melon, mulberry, strawberry, kiwi, water chestnut, asparagus, radish, rape, spinach, red beet, eggplant, tomato, cherry tomato, spinach, rape, water spinach, bitter gourd, cucumber, celery, mung bean, lotus leaf, kelp, laver, wakame, volvox, and spirulina; the vegetable and/or fruit of mild nature comprises at least one selected from the group consisted of: apple, cabbage, parsley, and Chinese cabbage.

5. The fruit-vegetable food of claim 1, wherein the vegetable is vegetable containing greater than or equal to 0.1% of glucosinolate in their dry weight.

6. The fruit-vegetable food of claim 1, wherein the probiotics comprise at least one selected from the group consisted of: Bifidobacterium adolescentis, Lactobacillus Bifidobacterium, Bifidobacterium bifidum, Lactobacillus crispatus, Lactobacillus acidophilus, Lactobacillus casei, Bifidobacterium longum, Bifidobacterium breve, Lactobacillus acidophilus, Lactobacillus bulgaricus, Lactobacillus fermenti, Lactobacillus gasseri, Lactobacillus helveticus, Lactobacillus johnsonii, Lactobacillus plantarum, Lactobacillus reuteri, Lactobacillus rhamnosus, Lactobacillus salivarius, and Streptococcus thermophilus.

7. The fruit-vegetable food of claim 1, wherein the food additive comprises at least one selected from the group consisted of: flavoring agent, essence, and thickener; the excipient comprises at least one selected from the group consisted of: filler, lubricant, and adhesive.

8. The fruit-vegetable food of claim 7, wherein the flavoring agent comprises at least one selected from the group consisted of: citric acid, vitamin C, tartaric acid, white sugar, stevioside, xylitol, and aspartame; the essence comprises at least one selected from the group consisted of: menthol-like flavor, strawberry flavor, banana flavor, pineapple flavor, melon flavor, and vanilla flavor; the thickener is carboxmethylcellulose; the filler comprises at least one selected from the group consisted of: maltodextrin, microcrystalline cellulose, lactose, tapioca starch, and corn starch; the lubricant comprises at least one selected from the group consisted of magnesium stearate and talcum powder; the adhesive is syrup.

9. Method for preparing the fruit-vegetable food for treating oral ulcer of claim 1, comprising:
selecting the vegetable and/or fruit according to claim 1;
adding food additive, excipient, and probiotic powder; and
mixing evenly to obtain the fruit-vegetable food.

10. The method for preparing the fruit-vegetable food for treating oral ulcer of claim 9, further comprising a step of preparing the fruit-vegetable food into a form of tablet, electuary, solid drink or vegetable paper.
